(19) United States
(12) Patent Application Publication
(10) Pub. No.: US 2022/0118439 A1
(43) Pub. Date: Apr. 21, 2022

(54) METHOD OF MOLDING AND MANUFACTURING FOR FLUIDIC DEVICE REAGENT TESTING CARTRIDGE AND PODS

(71) Applicant: Enplas Corporation, Kawaguchi-shi (JP)

(72) Inventor: Daisuke MORIKOSHI, Kawaguchi-shi (JP)

(21) Appl. No.: 17/487,814

(22) Filed: Sep. 28, 2021

Related U.S. Application Data
(63) Continuation-in-part of application No. 17/072,269, filed on Oct. 16, 2020, now abandoned.

(51) Int. Cl. B01L 3/00

(52) U.S. Cl. CPC B01L 3/502; B01L 3/508; B01L 2200/025; B01L 2200/04; B01L 2200/0689; B01L 2400/049; B01L 2200/141; B01L 2300/042; B01L 2300/0609; B01L 2300/105; B01L 2200/16

(57) ABSTRACT

A cartridge for detecting one or more characteristics of a fluid in a diagnostic assay system includes a plurality of chambers. At least one of the plurality of chambers includes a wall. The wall includes an intermediate portion disposed between an exterior surface and an interior surface of the wall. The intermediate portion can include a hygroscopic element configured to extend an active period of detection of a reagent. The intermediate portion can include a dummy material configured to slow a wetting of a reagent by the fluid. The intermediate portion can include an air gap configured to reduce internal stress and/or strain in the cartridge. A reagent can be enclosed by the wall of the at least one of the plurality of chambers. The reagent can be a lyophilized reagent.

### CROSS REFERENCE TO RELATED APPLICATIONS

This application is a continuation-in-part of U.S. application Ser. No. 17/072,269, filed Oct. 16, 2020, which is hereby incorporated herein in its entirety by reference for all purposes.

### FIELD

The present disclosure relates to cartridges, for example, disposable assay cartridges and related apparatuses, systems, and methods for making and using such cartridges. Example applications for related cartridges include uses in portable assay systems for fluidic reagent testing.

### BACKGROUND

Fluid analysis of biological substances for assay testing and detection can require a series of processing steps. These processing steps can require that a particular fluid or fluids contact a reaction area at different times and in varying succession. A single fluid sample can be subjected to a variety of processes or steps prior to contact with a reaction area.

A recent development in the field of diagnostic testing includes a portable diagnostic fluidic device capable of performing a variety of procedures normally performed manually by technicians in a laboratory. A portable diagnostic fluidic device can include a base unit for receiving a cartridge for conducting an assay test. For example, cartridges can be prepared for testing blood-borne diseases, food-borne bacteria, microorganisms such as bacteria or viruses, etc.

In a fluidic device in which a plurality of such processing steps are continuously performed, a cartridge can include a plurality of chambers each containing a reagent used in the assay test. A reagent that is unstable over time can be dehydrated or lyophilized to maximize shelf life and reliability. The lyophilized reagent can be stored within a chamber or multiple chambers of the cartridge and can be dissolved in a fluid immediately before use.

However, a lyophilized reagent is vulnerable to fluids and moisture in the cartridge. For example, a chamber containing a fluid can affect an adjacent chamber containing a lyophilized reagent that absorbs the fluid and/or associated fluid moisture. Also, molded cartridges are vulnerable to cracks and defects depending on the chamber thickness. Due to these vulnerabilities, any manufacturing defects in conventional cartridges, environmental conditions, or a combination thereof, can cause lyophilized reagents or assay materials to be diluted, contaminated, eroded, or otherwise corrupted in ways that can reduce or eliminate their efficacy over time. Such problems can result in economic losses for testing facilities that need to dispose of materials that become unusably corrupted before they can be deployed. Additionally, if such compromised materials are used for actual testing, potentially inaccurate and unreliable test results may ensue. This may in turn have further ramifications for medical testing and treatment of individual patients, as well as potential implications for public health concerns on a larger scale.

### BRIEF SUMMARY

Accordingly, there is a need to stably store reagents (e.g., lyophilized reagents) in a plurality of chambers of an assay cartridge, extend an active period of detection of the reagents, slow a wetting of the reagents by a fluid, and reduce molding defects (e.g., vacuum voids) in the assay cartridge.

In some embodiments, a cartridge for detecting one or more characteristics of a fluid in a diagnostic assay system includes a plurality of chambers. At least one of the plurality of chambers includes a wall. The wall includes an intermediate portion disposed between an exterior surface and an interior surface of the wall.

In some embodiments, the intermediate portion includes a hygroscopic element configured to extend an active period of detection of a reagent. In some embodiments, the hygroscopic element includes a desiccant, a dehumidifying material, a silica gel, calcium oxide, calcium chloride, a zeolite, charcoal, or any combination thereof.

In some embodiments, the intermediate portion includes a dummy material configured to slow a wetting of a reagent by the fluid. In some embodiments, the dummy material includes a monohydrate, a disaccharide, a sugar, trehalose, sucrose, glucose, or any combination thereof.

In some embodiments, the intermediate portion is configured to reduce internal stress and/or strain in the cartridge. For example, the intermediate portion can include an air gap configured to reduce molding defects (e.g., vacuum voids, air pockets, sinks, cracks, fissures, etc.) in the cartridge due to internal stress and/or strain in the cartridge.

In some embodiments, the intermediate portion includes an inert gas. In some embodiments, the inert gas can include a noble gas, dinitrogen (N₂), carbon dioxide (CO₂), or a combination thereof.

In some embodiments, the intermediate portion is uniform in width. In some embodiments, the width of the intermediate portion is between 1.0 mm and 5.0 mm. In some embodiments, the width of the intermediate portion is between 10% and 90% of a width of the wall.

In some embodiments, a subset of the plurality of chambers include a wall, and the wall corresponding to each chamber of the subset includes an intermediate portion disposed between an exterior surface and an interior surface of the wall. In some embodiments, each intermediate portion includes a hygroscopic element, a dummy material, an air gap, an inert gas, or any combination thereof.

In some embodiments, the cartridge further includes a rotor configured to rotate the plurality of chambers about a rotation axis.

In some embodiments, the cartridge further includes a reagent enclosed by the wall of the at least one of the plurality of chambers. In some embodiments, the reagent is a lyophilized reagent. In some embodiments, the cartridge further includes a lid coupled to the plurality of chambers and configured to seal the plurality of chambers.

In some embodiments, the fluid is enclosed in one of the plurality of chambers that is external to the intermediate portion and external to the chamber enclosing the reagent.

In some embodiments, a diagnostic assay system includes a cartridge, a syringe system, a first actuator, a second actuator, and a controller. The cartridge is configured to receive a fluid and includes a plurality of chambers and a rotor. The rotor includes a rotor port in fluid communication with at least one of the plurality of chambers. The syringe system is coupled to the cartridge and is configured to provide the fluid to the rotor port. The syringe system includes a syringe barrel having a barrel port and a syringe plunger configured to inject and withdraw the fluid. The first actuator is coupled to the cartridge and is configured to rotate the rotor port into alignment with the barrel port. The second actuator is coupled to the syringe system and is configured to translate the syringe plunger. The controller is coupled to the first and second actuators and is configured to detect one or more characteristics of the fluid based on an interaction of the fluid with one or more lyophilized reagents. One of the plurality of chambers includes a wall enclosing a lyophilized reagent. The wall includes an intermediate portion disposed between an exterior surface and an interior surface of the wall.

In some embodiments, the intermediate portion includes a hygroscopic element configured to extend an active period of detection of the reagent. In some embodiments, the intermediate portion includes a dummy material configured to slow a wetting of the reagent by the fluid. In some embodiments, the intermediate portion is configured to reduce internal stress and/or strain in the cartridge. For example, the intermediate portion can include an air gap configured to reduce molding defects (e.g., vacuum voids, air pockets, sinks, cracks, fissures, etc.) in the cartridge due to internal stress and/or strain in the cartridge. In some embodiments, the intermediate portion includes an inert gas. In some embodiments, the intermediate portion is configured to decrease contamination in the cartridge. For example, the intermediate portion can include an inert gas configured to decrease contamination in the cartridge.

In some embodiments, a method of detecting one or more characteristics of a fluid in a diagnostic assay system includes forming a cartridge having a plurality of chambers and a rotor. A first chamber of the plurality of chambers includes a wall having an intermediate portion disposed between an exterior surface and an interior surface of the wall. In some embodiments, the method further includes disposing a reagent within the first chamber. In some embodiments, the method further includes causing the fluid to interact with the reagent in the first chamber. In some embodiments, the intermediate portion includes an inert gas.

In some embodiments, the method further includes disposing a hygroscopic element, a dummy material, air, or an inert gas in the intermediate portion in order to extend an active period of interacting the fluid with the reagent. In some embodiments, the method further includes detecting one or more characteristics of the fluid based on the interaction of the fluid with the reagent.

In some embodiments, the method further includes interacting the fluid with a second reagent in a second chamber of the plurality of chambers. The second chamber includes a wall having a hygroscopic element, a dummy material, an air gap, or an inert gas disposed between an exterior surface and an interior surface of the wall. In some embodiments, the method further includes detecting one or more characteristics of the fluid based on the interaction of the fluid with the reagent and the second reagent.

Implementations of any of the techniques described above may include an apparatus, a device, a system, a method, and/or a process. The details of one or more implementations are set forth in the accompanying drawings and the description below. Other features will be apparent from the description and drawings, and from the claims.

Further features and example aspects of the embodiments, as well as the structure and operation of various embodiments, are described in detail below with reference to the accompanying drawings. It is noted that the embodiments are not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS/FIGURES

The accompanying drawings, which are incorporated herein and form part of the specification, illustrate the embodiments and, together with the description, further serve to explain the principles of the embodiments and to enable a person skilled in the relevant art(s) to make and use the embodiments.

FIG. 1 is a schematic perspective illustration of a diagnostic assay system with an assay cartridge, according to an example embodiment.

FIG. 2 is a partial schematic perspective exploded illustration of the diagnostic assay system and assay cartridge of FIG. 1, according to an example embodiment.

FIG. 3 is a schematic bottom perspective illustration of the assay cartridge of FIGS. 1 and 2, according to an example embodiment.

FIG. 4 is a schematic top perspective illustration of the assay cartridge shown in FIG. 3, according to an example embodiment.

FIG. 5 is a schematic top plan expanded illustration of a pair of chambers with intermediate portions shown in FIG. 4, according to an example embodiment.

FIG. 6 is a schematic top plan expanded illustration of a chamber with an intermediate portion shown in FIG. 4, according to an example embodiment.

FIG. 7 illustrates a flow diagram for detecting one or more characteristics of a fluid, according to an example embodiment.

The features and example aspects of the embodiments will become more apparent from the detailed description set forth below when taken in conjunction with the drawings, in which like reference characters identify corresponding elements throughout. In the drawings, like reference numbers generally indicate identical, functionally similar, and/or structurally similar elements. Additionally, generally, the left-most digit(s) of a reference number identifies the drawing in which the reference number first appears. Unless otherwise indicated, the drawings provided throughout the disclosure should not be interpreted as to-scale drawings.

### DETAILED DESCRIPTION

This specification discloses one or more embodiments that incorporate the features of this present invention. The disclosed embodiment(s) merely exemplify the present invention. The scope of the invention is not limited to the disclosed embodiment(s). The present invention is defined by the claims appended hereto.

The embodiment(s) described, and references in the specification to “one embodiment,” “an embodiment,” “an example embodiment,” etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is understood that it is within the knowledge of one skilled in the art to effect such feature, structure, or characteristic in connection with other embodiments whether or not explicitly described.

Spatially relative terms, such as “beneath,” “below,” “lower,” “above,” “on,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The term “about” or “substantially” or “approximately” as used herein indicates the value of a given quantity that can vary based on a particular technology. Based on the particular technology, the term “about” or “substantially” or “approximately” can indicate a value of a given quantity that varies within, for example, 1-15% of the value (e.g., ±1%, ±2%, ±5%, ±10%, or ±15% of the value).

The term “air gap” as used herein indicates a gap or void in a material that can be filled with a gas (e.g., air, an inert gas, a noble gas, dinitrogen (N₂), carbon dioxide (CO₂)) or be empty (e.g., vacuum).

The term “inert gas” as used herein indicates a gas that does not undergo chemical reactions under a set of given conditions. An inert gas is not necessarily elemental and can be a compound gas (e.g., carbon dioxide (CO₂)).

The term “noble gas” as used herein indicates odorless, colorless, monatomic gases with low chemical reactivity including helium (He), neon (Ne), argon (Ar), krypton (Kr), xenon (Xe), and radon (Rn).

Embodiments of the disclosure may be implemented in hardware, firmware, software, or any combination thereof. Embodiments of the disclosure may also be implemented as instructions stored on a machine-readable medium, which may be read and executed by one or more processors. A machine-readable medium may include any mechanism for storing or transmitting information in a form readable by a machine (e.g., a computing device). For example, a non-transitory machine-readable storage medium may include read-only memory (ROM); random-access memory (RAM); magnetic storage media, optical storage media, flash memory (solid-state) devices, or any combination thereof. Transmission media may include electrical, optical, acoustical, or other forms of propagated signals (e.g., carrier waves, infrared signals, digital signals, etc.), and other signals. Further, firmware, software, routines, and/or instructions may be described herein as performing certain actions. However, it should be appreciated that such descriptions are merely for convenience and that such actions in fact result from computing devices, processors, controllers, or other devices executing the firmware, software, routines, instructions, etc.

Example Diagnostic Assay Systems

As discussed above, fluid analysis of biological substances (e.g., blood, proteins, DNA, etc.) for assay testing and detection may involve any steps in a series of processing steps (e.g., mixing, heating, cooling, detection, etc.). These processing steps may involve subjecting a particular fluid or fluids to contact with a reaction area at different times and in varying succession. A single fluid sample can be subjected to a variety of steps prior to contact with a reaction area.

A portable diagnostic fluidic device may be configured to perform a variety of procedures typically performed manually by technicians in a laboratory, in some use cases. A portable diagnostic fluidic device can include a base unit for receiving a cartridge for conducting an assay test. For example, cartridges can be prepared for testing blood-borne diseases, food-borne bacteria, or microorganisms such as bacteria or viruses, etc.

In a fluidic device in which a plurality of such processing steps are continuously performed, a cartridge can include a plurality of chambers each containing a reagent used in the assay test (e.g., polymerase chain reaction (PCR) primers, enzymes, and/or certain chemical compounds). A reagent that is unstable over time may be dehydrated or lyophilized (e.g., freeze-dried, cryodesiccated, etc.) to improve shelf life and reliability over time, reducing a rate of natural degradation. The lyophilized reagent can be stored within a chamber or chambers of the cartridge and can be dissolved in a fluid immediately before use.

However, a lyophilized reagent can be vulnerable to fluids and moisture in the cartridge. For example, a chamber containing a fluid can affect (e.g., contaminate) an adjacent chamber containing a lyophilized reagent that absorbs the fluid and/or associated fluid moisture. Also, molded cartridges, such as those formed by injection molding, are vulnerable to cracks and defects (e.g., vacuum voids) depending on the chamber thickness.

Embodiments of cartridge apparatuses and diagnostic assay systems as discussed below may stably store reagents (e.g., lyophilized reagents) in a plurality of chambers of an assay cartridge, extend an active period of detection of the reagents, slow a wetting of the reagents by a fluid, and reduce molding defects (e.g., vacuum voids) in the cartridge.

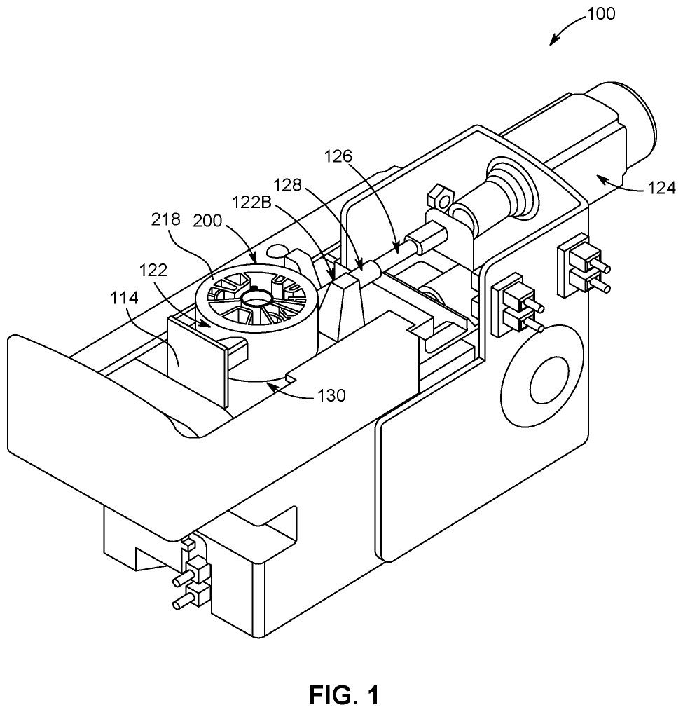

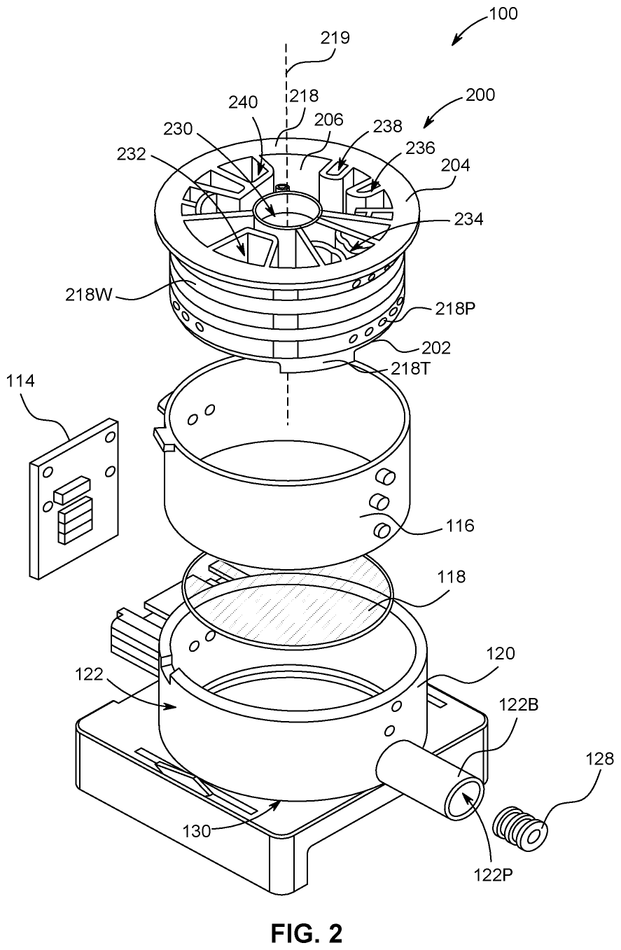

FIGS. 1 and 2 illustrate diagnostic assay system 100 with assay cartridge 200, according to various example embodiments. FIG. 1 is a schematic perspective illustration of diagnostic assay system 100 with assay cartridge 200, according to an example embodiment. FIG. 2 is a partial schematic perspective exploded illustration of diagnostic assay system 100 and assay cartridge 200 of FIG. 1, according to an example embodiment. Diagnostic assay system 100 can be configured to detect one or more characteristics of a fluid. Diagnostic assay system 100 can be further configured to control one or more fluids into and out of one or more peripheral chambers 232, 234, 236, 238, 240 of assay cartridge 200. In some embodiments, diagnostic assay system 100 may be a portable and compact (e.g., handheld) unit configured to receive one or more disposable assay cartridges 200 for different fluidic diagnostic tests. For example, each disposable assay cartridges 200 can be selectively configured for detecting a particular characteristic of a fluid (e.g., a marker for a blood, food, or biological disease, bacteria, and/or virus).

As shown in FIGS. 1 and 2, diagnostic assay system 100 can include assay cartridge 200, syringe system 122, first actuator 130 (e.g., rotary actuator), second actuator 124 (e.g., linear actuator), and controller 114. In some embodiments, diagnostic assay system 100 can detect one or more characteristics of a fluid based on an interaction of the fluid with a reagent (e.g., lyophilized reagent 260-264) of one or more peripheral chambers 232, 234, 236, 238, 240 of assay cartridge 200. For example, controller 114 of diagnostic assay system 100 can detect an interaction (e.g., chemically, optically, thermally, mechanically, magnetically, acoustically, etc.) of a fluid from syringe system 122 with one or more reagents (e.g., lyophilized reagent 260-264) of one or more peripheral chambers 232, 234, 236, 238, 240 of assay cartridge 200 and determine one or more characteristics (e.g., markers, DNA, cDNA, RNA, diseases, bacteria, viruses, etc.) of the fluid based on the detected interaction.

Assay cartridge 200 can be configured to provide one or more reagents (e.g., lyophilized reagent 260-264) in one or more peripheral chambers 232, 234, 236, 238, 240 for detecting one or more characteristics of a fluid in diagnostic assay system 100. As shown in FIG. 2, assay cartridge 200 can include bottom surface 202, top surface 204, sidewall surface 206, central chamber 230, one or more peripheral chambers 232, 234, 236, 238, 240, and rotor 218. In some embodiments, one or more peripheral chambers 232, 234, 236, 238, 240 can enclose and isolate a reagent (e.g., lyophilized reagent 260-264) for one or more different fluidic diagnostic tests.

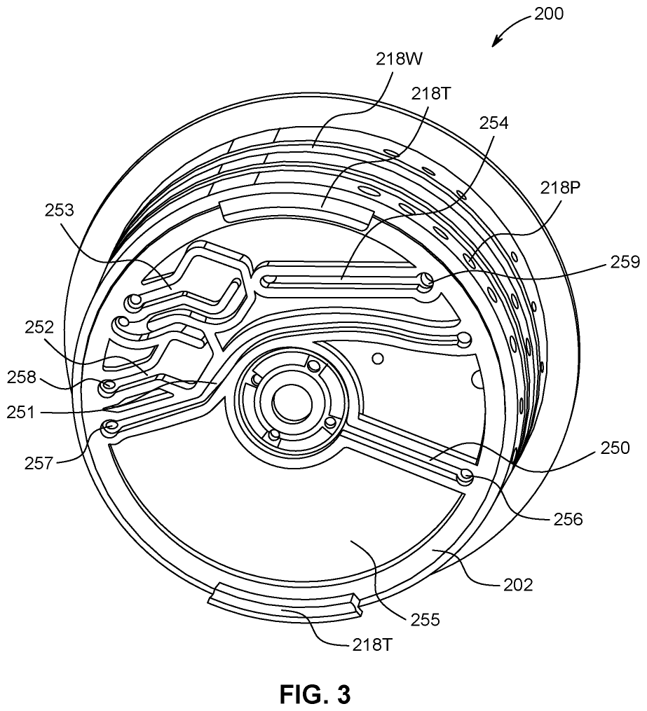

Rotor 218 can be configured to rotate the plurality of chambers 230, 232, 234, 236, 238, 240 about a rotation axis 219. Rotor 218 can be further configured to couple to syringe system 122 and first actuator 130 (e.g., rotary actuator) in order to be rotated about rotation axis 219. In some embodiments, rotor 218 can be a cylindrical rotor. Rotor 218 can include one or more rotor ports 218P, one or more rotor grooves 218W, and/or one or more rotor tabs 218T. In some embodiments, rotor ports 218P can be in fluid communication with one or more of the plurality of chambers 230, 232, 234, 236, 238, 240. For example, as shown in FIG. 3, rotor ports 218P can be in fluid communication with one or more channels 250, 251, 252, 253, 254 and/or channel ports 256, 257, 258, 259. In some embodiments, rotor grooves 218W and/or rotor tabs 218T can be coupled to first actuator 130 (e.g., rotary actuator) to rotate (e.g., incrementally, sequentially, etc.) rotor 218 about rotation axis 219. For example, one or more rotor ports 218P can be sequentially rotated (e.g., in a predetermined sequence) into alignment with barrel port 122P of syringe system 122. Details and various embodiments of assay cartridge 200 will be further discussed below with reference to FIGS. 3-6.

Syringe system 122 can be configured to provide a fluid to assay cartridge 200. In some embodiments, syringe system 122 can be coupled to assay cartridge 200 and provide a fluid to one or more rotor ports 218P. As shown in FIG. 2, syringe system 122 can include inner cartridge holder 116 (e.g., a hollow cylinder), cartridge seal plate 118 (e.g., a glass disc), outer cartridge holder 120 with syringe barrel 122B and barrel port 122P, plunger shaft 126, and syringe plunger 128 (e.g., an elastomer). In some embodiments, syringe system 122 can be coupled to assay cartridge 200, first actuator 130, second actuator 124, and controller 114 for multiple fluidic tests.

Inner cartridge holder 116 can be configured to support assay cartridge 200 and to allow rotation of rotor 218 about rotation axis 219. In some embodiments, inner cartridge holder 116 can be coupled to assay cartridge 200 and provide a stationary support for assay cartridge 200. For example, as shown in FIG. 2, inner cartridge holder 116 can be a hollow cylinder that is coupled to outer cartridge holder 120 and supports top surface 204 (e.g., a flange) of rotor 218 allowing rotor 218 to rotate about rotation axis 219 relative to outer cartridge holder 120.

Cartridge seal plate 118 can be configured to seal bottom surface 202 of assay cartridge 200. In some embodiments, cartridge seal plate 118 can contact bottom surface 202 to form a fluid tight (e.g., watertight) seal. For example, as shown in FIGS. 2 and 3, cartridge seal plate 118 may be a disc (e.g., a glass) that is coupled to bottom surface 202 and form a fluid tight seal with channels 250, 251, 252, 253, 254, bottom panel 255, and channel ports 256, 257, 258, 259 disposed (e.g., recessed) on bottom surface 202. Cartridge seal plate 118 may be formed from or otherwise made of an inert material relative to the fluid and/or reagent, such as glass, titanium, stainless steel, etc., any combination thereof, or another material having a surface coating or treatment to render it inert at the surface, such as polyethylene, polytetrafluoroethylene (PTFE), etc., or any combination thereof.

Outer cartridge holder 120 can be configured to support inner cartridge holder 116 and assay cartridge 200. Outer cartridge holder 120 can be further configured to provide a fluid to assay cartridge 200 via barrel port 122P of syringe barrel 122B. As shown in FIGS. 1 and 2, syringe plunger 128 can be coupled to barrel port 122P and plunger shaft 126 can be coupled to second actuator 124 in order to move a fluid into and out of one or more rotor ports 218P. In some embodiments, plunger shaft 126 can displace (e.g., translate) syringe plunger 128 (e.g., an elastomer) with syringe barrel 122B to move and/or admix one or more fluids contained in one or more peripheral chambers 232, 234, 236, 238, 240.

First actuator 130 can be configured to rotate one or more rotor ports 218P of rotor 218 into alignment with barrel port 122P of syringe barrel 122B. In some embodiments, first actuator 130 can include a rotary actuator (e.g., mechanical, electric, electromechanical, pneumatic, hydraulic, spring, shape memory, supercoiled polymer, vacuum, piezoelectric, stepper motor, servomotor, torque motor, etc.). For example, rotary actuator 130 can be coupled to rotor grooves 218W and/or rotor tabs 218T to rotate (e.g., incrementally, sequentially, etc.) rotor 218 about rotation axis 219. For example, one or more rotor ports 218P can be sequentially rotated (e.g., in a predetermined sequence) by first actuator 130 (e.g., a rotary actuator) into alignment with barrel port 122P of syringe system 122.

Second actuator 124 can be configured to translate syringe plunger 128 of syringe system 122 to inject and withdraw a fluid in one or more rotor ports 218P. In some embodiments, second actuator 124 can include a linear actuator (e.g., mechanical, electric, electromechanical, pneumatic, hydraulic, spring, shape memory, supercoiled polymer, vacuum, piezoelectric, stepper motor, servomotor, linear motor, etc.). For example, linear actuator 124 can be coupled to syringe plunger 128 via plunger shaft 126 to translate syringe plunger 128 towards and away from syringe barrel 122B in order to inject and withdraw a fluid in barrel port 122P and one or more rotor ports 218P. In some embodiments, second actuator 124 can generate a pressure (e.g., positive, negative (vacuum)) in syringe barrel 122B.

Controller 114 can be configured to detect one or more characteristics of a fluid based on an interaction of the fluid with one or more reagents (e.g., lyophilized reagent 260-264) within peripheral chambers 232, 234, 236, 238, 240. In some embodiments, controller 114 can include one or more computer processors (e.g., central processing unit (CPU)), microcontroller unit (MCU), etc., and/or one or more sensors (e.g., chemical sensor, optical sensor, thermal sensor, mechanical sensor, magnetic sensor, acoustic sensor, etc.), or other dedicated application-specific electronics or similar circuitry. In some embodiments, controller 114 can be coupled to first and second actuators 130, 124. For example, controller 114 can be coupled (e.g., electrically, wirelessly, mechanically, etc.) to rotary actuator 130 and control rotation of rotor 218 about rotation axis 219 relative to syringe system 122, and be coupled (e.g., electrically, wirelessly, mechanically, etc.) to linear actuator 124 and control translation of syringe plunger 128 towards and away from syringe barrel 122B.

In some embodiments, controller 114 can detect an interaction (e.g., chemically, optically, thermally, mechanically, magnetically, acoustically, etc.) of a fluid from syringe system 122 with one or more reagents (e.g., lyophilized reagent 260-264) of one or more peripheral chambers 232, 234, 236, 238, 240 of assay cartridge 200. For example, controller 114 can determine one or more characteristics (e.g., markers, DNA, cDNA, RNA, diseases, bacteria, viruses, etc.) of the fluid based on the detected interaction. In some embodiments, one or more characteristics of a fluid can be detected by controller 114 based on a first interaction of the fluid with a first reagent (e.g., lyophilized reagent 260-264) of one or more peripheral chambers 232, 234, 236, 238, 240 and a second interaction of the fluid with a second reagent (e.g., lyophilized reagent 260-264) of one or more peripheral chambers 232, 234, 236, 238, 240. For example, the fluid can interact with first lyophilized reagent 262 of peripheral chamber 236 and with second lyophilized reagent 263 of peripheral chamber 238. Controller 114 may detect one or more characteristics of the fluid. These characteristics as detected may be based at least in part on a result of either interaction or both interactions.

In some embodiments, assay cartridge 200 can include a lid (e.g., similar to cartridge seal plate 118) configured to seal top surface 204 of assay cartridge 200 and seal one or more peripheral chambers 232, 234, 236, 238, 240. In some embodiments, the lid (e.g., similar to cartridge seal plate 118) can contact top surface 204 to form a fluid tight (e.g., watertight) seal of one or more peripheral chambers 232, 234, 236, 238, 240. In some embodiments, the lid can include a sealing material (e.g., a metal foil, a heat seal, a seal plate, an epoxy seal, etc.).

Example Cartridge Apparatuses

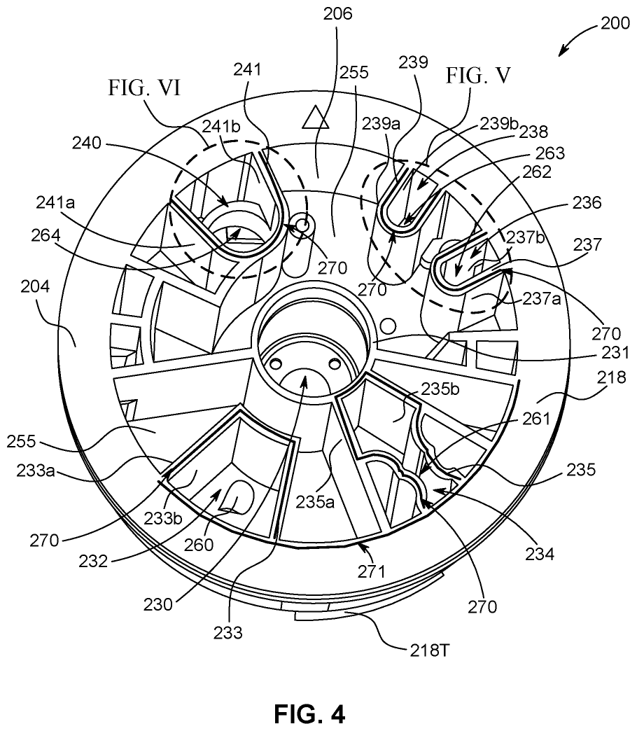

As shown in FIGS. 1 and 2, diagnostic assay system 100 can include assay cartridge 200. FIGS. 3-6 illustrate assay cartridge 200, according to various example embodiments. FIG. 3 is a schematic bottom perspective illustration of assay cartridge 200 of FIGS. 1 and 2, according to an example embodiment. FIG. 4 is a schematic top perspective illustration of assay cartridge 200 of FIG. 3, according to an example embodiment.

Assay cartridge 200 can be configured to provide one or more reagents (e.g., lyophilized reagent 260-264) in one or more peripheral chambers 232, 234, 236, 238, 240 for detecting (e.g., chemically, optically, thermally, mechanically, magnetically, acoustically, etc.) one or more characteristics (e.g., markers, DNA, cDNA, RNA, diseases, bacteria, viruses, etc.) of a fluid in diagnostic assay system 100. In some embodiments, one or more peripheral chambers 232, 234, 236, 238, 240 can enclose and isolate one or more reagents (e.g., lyophilized reagent 260-264) for one or more different fluidic diagnostic tests.

As shown in FIGS. 3 and 4, assay cartridge 200 can include bottom surface 202, top surface 204, sidewall surface 206, central chamber 230, one or more peripheral chambers 232, 234, 236, 238, 240, channels 250, 251, 252, 253, 254, bottom panel 255, channel ports 256, 257, 258, 259, and rotor 218 with rotor ports 218P, rotor grooves 218W, and rotor tabs 218T. In some embodiments, one or more peripheral chambers 232, 234, 236, 238, 240 can include an intermediate portion 270. For example, as shown in FIG. 4, any of peripheral chambers 232, 234, 236, 238, 240 may include a chamber wall 233, 235, 237, 239 with an intermediate portion 270, for example, an air gap, a hygroscopic element, a dummy material, or an inert gas. In some embodiments, assay cartridge 200 can include an intermediate wall, a gap, a material, or an inert gas (e.g., intermediate portion 270) in one or more portions of rotor 218. For example, as shown in FIG. 4, intermediate portion 271 (e.g., similar to intermediate portion 270) can be disposed between an interior radial circumference and an exterior radial circumference of sidewall surface 206 and between top surface 204 and bottom panel 255.

As shown in FIG. 3, channels 250, 251, 252, 253, 254, bottom panel 255, and channel ports 256, 257, 258, 259 can be disposed (e.g., recessed) in bottom surface 202 of assay cartridge 200. In some embodiments, cartridge seal plate 118 can form a fluid tight seal (e.g., watertight) with channels 250, 251, 252, 253, 254, bottom panel 255, and channel ports 256, 257, 258, 259 for transferring a fluid to one or more peripheral chambers 232, 234, 236, 238, 240. In some embodiments, channels 250, 251, 252, 253, 254 can be fluidly coupled to channel ports 256, 257, 258, 259. For example, as shown in

FIG. 3, channel 250 can be fluidly connected to channel port 256, channel 251 can be fluidly connected to channel port 257, and channel 252 can be fluidly connected to channel 258. In some embodiments, one or more peripheral chambers 232, 234, 236, 238, 240 can be in fluid communication with one or more rotor ports 218P. For example, as shown in FIG. 3, one or more channel ports 256, 257, 258, 259 can be in fluid communication with one or more rotor ports 218P and transfer a fluid via channels 250, 251, 252, 253, 254 and/or bottom panel 255 to one or more peripheral chambers 232, 234, 236, 238, 240.

As shown in FIG. 4, assay cartridge 200 can include one or more peripheral chambers 232, 234, 236, 238, 240 distributed around central chamber 230. Peripheral chambers 232, 234, 236, 238, 240 can be configured to enclose one or more reagents to provide a reaction area with a fluid for diagnostic fluidic testing. In some embodiments, peripheral chambers 232, 234, 236, 238, 240 can be disposed between bottom surface 202 and top surface 204. For example, as shown in FIG. 4, peripheral chambers 232, 234, 236, 238, 240 can be disposed between bottom panel 255 (e.g., recess in bottom surface 202 to form a fluid channel) and top surface 204.

In some embodiments, one or more reagents 260, 261, 262, 263, 264 can be disposed within peripheral chambers 232, 234, 236, 238, 240, respectively. For example, as shown in FIG. 4, each peripheral chamber 232, 234, 236, 238, 240 can enclose a reagent 260, 261, 262, 263, 264, respectively. In some embodiments, one or more reagents 260, 261, 262, 263, 264 can be a lyophilized reagent. For example, as shown in FIGS. 4 and 6, lyophilized reagent 264 can be disposed in peripheral chamber 240 within interior wall surface 24 lb. In some embodiments, central chamber 230 can receive a fluid from syringe system 122 and transfer the fluid to one or more peripheral chambers 232, 234, 236, 238, 240 with one or more reagents (e.g., lyophilized reagent 260-264) for diagnostic fluidic testing. In some embodiments, one or more peripheral chambers 232, 234, 236, 238, 240 can be open (e.g., contain no reagent). For example, one or more open peripheral chambers 232, 234, 236, 238, 240 can receive a mixed fluid-reagent for a subsequent diagnostic fluidic test and/or for detection of the mixed fluid-reagent by controller 114 (e.g., via a sensor).

In some embodiments, a reagent (e.g., lyophilized reagent 260-264) of one or more peripheral chambers 232, 234, 236, 238, 240 can interact with a fluid. For example, a fluid can be disposed in central chamber 230 and/or one or more cartridge ports 218P and be distributed to one or more peripheral chambers 232, 234, 236, 238, 240 via one or more channels 250, 251, 252, 253 and/or one or more channel ports 256, 257, 258, 259. In some embodiments, a fluid can interact with a second reagent (e.g., lyophilized reagent 260-264) in a second peripheral chamber 232, 234, 236, 238, 240. For example, as shown in FIGS. 3 and 4, a fluid can interact with peripheral chamber 238 with lyophilized reagent 263 and peripheral chamber 240 via channels 253, 254 and channel port 259.

As shown in FIG. 4, peripheral chambers 232, 234, 236, 238, 240 can be separated and/or surrounded by chamber walls 233, 235, 237, 239, 241. Chamber walls 233, 235, 237, 239, 241 can be configured to define part of peripheral chambers 232, 234, 236, 238, 240. In some embodiments, one or more chamber walls 233, 235, 237, 239, 241 can be coupled to sidewall surface 206. In some embodiments, as shown in FIG. 4, chamber walls 233, 235, 237, 239, 241 can be radial, circumferential, and/or U-shaped walls. In some embodiments, one or more chamber walls 233, 235, 237, 239, 241 can have a U-shape having an arcuate portion disposed between two legs coupled to rotor 218. For example, as shown in FIGS. 4-6, free ends of both legs of the U-shape can be coupled to sidewall surface 206 between top surface 204 and bottom panel 255.

As shown in FIG. 4, one or more chamber walls 231, 233, 235, 237, 239 of peripheral chambers 232, 234, 236, 238, 240 can include an intermediate portion 270. Intermediate portion 270 can be configured to extend an active period of detection of a reagent (e.g., lyophilized reagent 260-264). Intermediate portion 270 can be further configured to slow a wetting of a reagent (e.g., lyophilized reagent 260-264) by a fluid. For example, the wetting of a lyophilized or dry reagent (e.g., lyophilized reagent 260-264) can be reduced or slowed compared to a solid chamber wall that does not include an intermediate wall, a gap, a material, or an inert gas (e.g., intermediate portion 270). Intermediate portion 270 can be further configured to reduce molding defects in array cartridge 200. For example, an intermediate gap (e.g., air gap 272) can reduce or mitigate adverse molding defects, such as vacuum voids, air pockets, sinks, cracks, fissures, etc., by reducing internal stresses and/or strains in a mold during manufacturing (e.g., molding). Further, an intermediate gap (e.g., air gap 272) can reduce a probability or number of incidences of molding defects (e.g., vacuum voids, air pockets, sinks, cracks, fissures, etc.) during manufacturing. Intermediate portion 270 can be disposed between an exterior wall surface 233a, 235a, 237a, 239a, 241a and an interior wall surface 233b, 235b, 237b, 239b, 241b of chamber walls 231, 233, 235, 237, 239, respectively. In some embodiments, a width of intermediate portion 270 can be uniform. For example, the width of intermediate portion 270 can be constant throughout a chamber wall 233, 235, 237, 239, 241. In some embodiments, a width of intermediate portion 270 can be at least 1.0 mm. For example, intermediate portion 270 can be about 1.0 mm to about 2.0 mm. In some embodiments, intermediate portion 270 can have a width between 1.0 mm and 5.0 mm. In some embodiments, intermediate portion 270 can have a width between 10% and 90% of a width of a respective chamber wall 231, 233, 235, 237, 239. For example, the width can be between 35% and 65% of the width of a respective chamber wall 231, 233, 235, 237, 239.

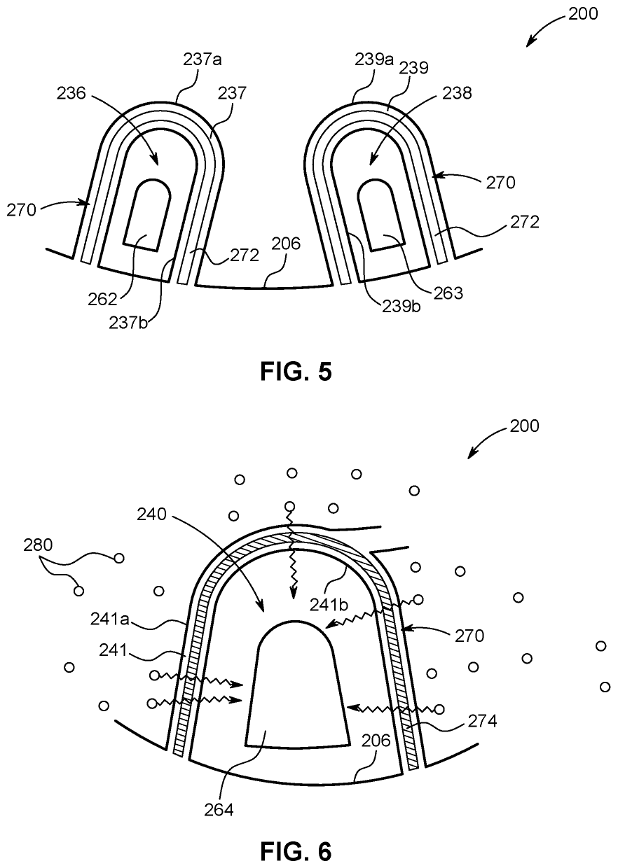

In some embodiments, as shown in FIG. 4, each chamber wall 231, 233, 235, 237, 239 of peripheral chambers 232, 234, 236, 238, 240 can include an intermediate portion 270. In some embodiments, intermediate portion 270 can include an air gap and/or a partition wall. For example, as shown in FIGS. 5 and 6, intermediate portion 270 can include air gap 272 (e.g., air, an inert gas) and/or partition wall 274 (e.g., a hygroscopic element, a dummy material). In some embodiments, one or more peripheral chambers 232, 234, 236, 238, 240 can include a chamber wall 233, 235, 237, 239, 241 with an air gap 272 disposed between an exterior wall surface 233a, 235a, 237a, 239a, 241a and an interior wall surface 233b, 235b, 237b, 239b, 241b, respectively. In some embodiments, one or more peripheral chambers 232, 234, 236, 238, 240 can include a chamber wall 233, 235, 237, 239, 241 with a partition wall 274 disposed between an exterior wall surface 233a, 235a, 237a, 239a, 241a and an interior wall surface 233b, 235b, 237b, 239b, 241b, respectively.

In some embodiments, a fluid for fluidic reagent testing can be enclosed in one of the peripheral chambers 232, 234, 236, 238, 240 that is external to intermediate portion 270 and external to chamber wall 233, 235, 237, 239, 241. For example, the fluid can be enclosed in peripheral chamber 236 that is external to intermediate portion 270 and external to chamber wall 239 of peripheral chamber 238.

FIG. 5 is a schematic top plan expanded illustration of peripheral chambers 236, 238 each with an intermediate portion 270 shown in FIG. 4, according to an example embodiment. As shown in FIG. 5, peripheral chambers 236, 238 can include chamber walls 237, 239 each with air gap 272 disposed between exterior wall surface 237a, 239a and interior wall surface 237b, 239b, respectively. In some embodiments, air gap 272 can extend through a height of the chamber wall 233, 235, 237, 239, 241. For example, as shown in FIGS. 4 and 5, air gap 272 can extend from top surface 204 to bottom panel 255 of assay cartridge 200.

FIG. 6 is a schematic top plan expanded illustration of peripheral chamber 240 with an intermediate portion 270 shown in FIG. 4, according to an example embodiment. As shown in FIG. 6, peripheral chamber 240 can include chamber wall 241 with partition wall 274 disposed between exterior wall surface 241a and interior wall surface 241b. In some embodiments, partition wall 274 can extend through a height of the chamber wall 233, 235, 237, 239, 241. For example, as shown in FIGS. 4 and 6, partition wall 274 can extend from top surface 204 to bottom panel 255 of assay cartridge 200.

As shown in FIG. 6, partition wall 274 can prevent or reduce external moisture 280 from penetrating an enclosed reagent (e.g., lyophilized reagent 264), which may in turn slow a wetting of the enclosed reagent and/or extends an active period of detection of the reagent. In some embodiments, partition wall 274 can be a hygroscopic element configured to extend an active period of interacting (e.g., exposing, reacting, contacting, evaporating, dissolving, etc.) a fluid with a reagent (e.g., lyophilized reagent 260-264). For example, partition wall 274 can include a desiccant, a dehumidifying material, a silica gel, calcium oxide, calcium chloride, a zeolite, charcoal, or some any combination thereof. In some embodiments, partition wall 274 can be a dummy material configured to slow a wetting of a reagent (e.g., lyophilized reagent 260-264) by a fluid. For example, partition wall 274 can include a monohydrate, a disaccharide, a sugar, trehalose, sucrose, glucose, or any combination thereof. In some embodiments, a partition wall 274 can be disposed in an air gap 272. For example, partition wall 274 can completely fill air gap 272. In some embodiments, at least one partition wall 274 can be disposed within an air gap 272, effectively forming multiple air gaps.

Example Flow Diagram

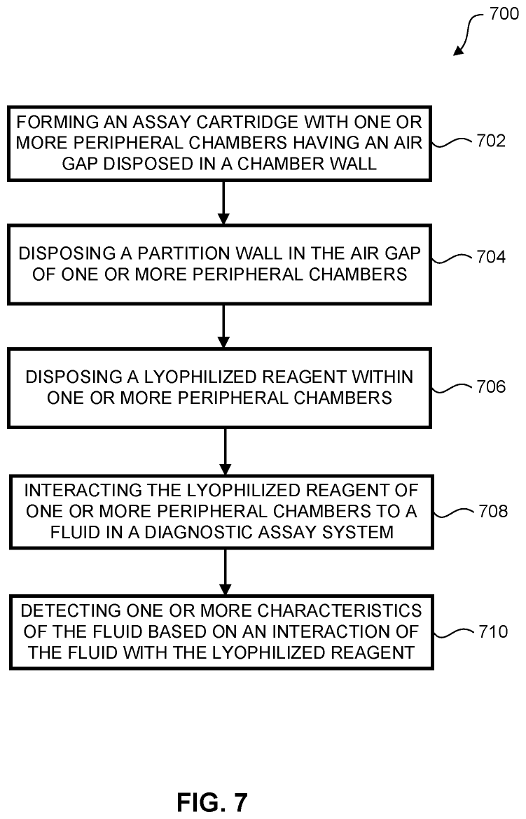

FIG. 7 illustrates flow diagram 700 for detecting one or more characteristics of a fluid in diagnostic assay system 100, according to an embodiment. It is to be appreciated that not all steps in FIG. 7 are needed to perform the disclosure provided herein. Further, some of the steps may be performed simultaneously, sequentially, and/or in a different order than shown in FIG. 7. Flow diagram 700 shall be described with reference to FIGS. 1-6. However, flow diagram 700 is not limited to those example embodiments.

In step 702, as shown in the example of FIGS. 4-6, assay cartridge 200 can be formed with central chamber 230 and one or more peripheral chambers 232, 234, 236, 238, 240 and rotor 218. For example, assay cartridge 200 can be formed by molding, thermoforming, or vacuum forming. In some embodiments, one or more peripheral chambers 232, 234, 236, 238, 240 can include a chamber wall 233, 235, 237, 239, 241 with an air gap 272 disposed between an exterior wall surface 233a, 235a, 237a, 239a, 241a and an interior wall surface 233b, 235b, 237b, 239b, 241b, respectively. For example, as shown in FIG. 5, peripheral chambers 236, 238 can include chamber walls 237, 239 each with air gap 272 disposed between exterior wall surface 237a, 239a and interior wall surface 237b, 239b, respectively, for at least some of the peripheral chambers. In some embodiments, one or more chamber walls 233, 235, 237, 239, 241 can have a U-shape having an arcuate portion disposed between two legs coupled to rotor 218. For example, as shown in FIGS. 4-6, free ends of both legs of the U-shape can be coupled to sidewall surface 206 between top surface 204 and bottom panel 255.

In step 704, as shown in the example of FIGS. 4-6, a partition wall 274 can be disposed in an air gap 272 of one or more peripheral chambers 232, 234, 236, 238, 240 of assay cartridge 200 formed in step 702. For example, as shown in FIG. 6, partition wall 274 can be disposed in air gap 272 of peripheral chamber 240 between exterior wall surface 241a and interior wall surface 241b. In some embodiments, partition wall 274 can be a hygroscopic element configured to extend an active period of interacting a fluid with a reagent (e.g., lyophilized reagent 260-264). For example, partition wall 274 can include a desiccant, a dehumidifying material, a silica gel, calcium oxide, calcium chloride, a zeolite, charcoal, or any combination thereof. In some embodiments, partition wall 274 can be a dummy material configured to slow a wetting of a reagent (e.g., lyophilized reagent 260-264) by a fluid. For example, partition wall 274 can include a monohydrate, a disaccharide, a sugar, trehalose, sucrose, glucose, or any combination thereof.

In step 706, as shown in the example of FIGS. 4-6, a reagent (e.g., lyophilized reagent 260-264) can be disposed within one or more peripheral chambers 232, 234, 236, 238, 240. For example, as shown in FIG. 6, lyophilized reagent 264 can be disposed in peripheral chamber 240 within interior wall surface 241b.

In step 708, as shown in the example of FIGS. 3 and 4, the reagent (e.g., lyophilized reagent 260-264) of one or more peripheral chambers 232, 234, 236, 238, 240 can interact with a fluid. For example, as shown in FIGS. 3 and 4, a fluid can be disposed in central chamber 230 and/or one or more cartridge ports 218P and be distributed to one or more peripheral chambers 232, 234, 236, 238, 240 via one or more channels 250, 251, 252, 253 and/or one or more channel ports 256, 257, 258, 259. In some embodiments, a fluid can interact with a second reagent (e.g., lyophilized reagent 260-264) in a second peripheral chamber 232, 234, 236, 238, 240. For example, as shown in FIGS. 3 and 4, a fluid can interact with peripheral chamber 238 with lyophilized reagent 263 and peripheral chamber 240 via channels 253, 254 and channel port 259.

In step 710, as shown in the example of FIGS. 1-4, one or more characteristics of a fluid can be detected based on the interaction of the fluid with the reagent (e.g., lyophilized reagent 260-264) of one or more peripheral chambers 232, 234, 236, 238, 240. For example, as shown in FIGS. 2 and 4, controller 114 of diagnostic assay system 100 can detect the interaction (e.g., chemically, optically, thermally, mechanically, magnetically, acoustically, etc.) of a fluid from syringe system 122 with one or more reagents (e.g., lyophilized reagent 260-264) of one or more peripheral chambers 232, 234, 236, 238, 240 of assay cartridge 200 and determine one or more characteristics (e.g., markers, DNA, cDNA, RNA, diseases, bacteria, viruses, etc.) of the fluid based on the detected interaction. In some embodiments, controller 114 can include one or more computer processors (e.g., central processing unit (CPU), microcontroller unit (MCU), etc.) and/or one or more sensors (e.g., chemical sensor, optical sensor, thermal sensor, mechanical sensor, magnetic sensor, acoustic sensor, etc.) or other dedicated application-specific electronics or similar circuitry.

In some embodiments, one or more characteristics of a fluid can be detected based on a first interaction of the fluid with a first reagent (e.g., lyophilized reagent 260-264) of one or more peripheral chambers 232, 234, 236, 238, 240 and a second interaction of the fluid with a second reagent (e.g., lyophilized reagent 260-264) of one or more peripheral chambers 232, 234, 236, 238, 240. For example, as shown in FIGS. 2, 4, and 5, a fluid can interact with first lyophilized reagent 262 of peripheral chamber 236 and with second lyophilized reagent 263 of peripheral chamber 238. Controller 114 of diagnostic assay system 100 may detect one or more characteristics of the fluid. These characteristics as detected may be based at least in part on a result of either interaction or both interactions.

It is to be understood that the phraseology or terminology herein is for the purpose of description and not of limitation, such that the terminology or phraseology of the present specification is to be interpreted by those skilled in relevant art(s) in light of the teachings herein.

The following examples are illustrative, but not limiting, of the embodiments of this disclosure. Other suitable modifications and adaptations of the variety of conditions and parameters normally encountered in the field, and which would be apparent to those skilled in the relevant art(s), are within the spirit and scope of the disclosure.

While specific embodiments have been described above, it will be appreciated that the embodiments may be practiced otherwise than as described. The description is not intended to limit the scope of the claims.

It is to be appreciated that the Detailed Description section, and not the Summary and Abstract sections, is intended to be used to interpret the claims. The Summary and Abstract sections may set forth one or more but not all example embodiments as contemplated by the inventor(s), and thus, are not intended to limit the embodiments and the appended claims in any way.

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the embodiments that others can, by applying knowledge within the skill of the art, readily modify and/or adapt for various applications such specific embodiments, without undue experimentation, without departing from the general concept of the embodiments. Therefore, such adaptations and modifications are intended to be within the meaning and range of equivalents of the disclosed embodiments, based on the teaching and guidance presented herein.

The breadth and scope of the embodiments should not be limited by any of the above-described example embodiments, but should be defined only in accordance with the following claims and their equivalents.

What is claimed is:

1. A cartridge for detecting one or more characteristics of a fluid in a diagnostic assay system, the cartridge comprising:
a plurality of chambers,
wherein at least one of the plurality of chambers comprises a wall, and
wherein the wall comprises an intermediate portion disposed between an exterior surface and an interior surface of the wall.

2. The cartridge of claim 1, wherein the intermediate portion comprises a hygroscopic element configured to extend an active period of detection of a reagent.

3. The cartridge of claim 2, wherein the hygroscopic element comprises a desiccant, a dehumidifying material, a silica gel, calcium oxide, calcium chloride, a zeolite, charcoal, or any combination thereof.

4. The cartridge of claim 1, wherein the intermediate portion comprises a dummy material configured to slow a wetting of a reagent by the fluid.

5. The cartridge of claim 4, wherein the dummy material comprises a monohydrate, a disaccharide, a sugar, trehalose, sucrose, glucose, or any combination thereof.

6. The cartridge of claim 1, wherein the intermediate portion is configured to reduce internal stress and/or strain in the cartridge. The cartridge of claim 1, wherein the intermediate portion is uniform in width.

8. The cartridge of claim 7, wherein the width of the intermediate portion is between 10% and 90% of a width of the wall.

9. The cartridge of claim 1, wherein:
a subset of the plurality of chambers comprises a wall, and
the wall corresponding to each chamber of the subset comprises an intermediate portion disposed between an exterior surface and an interior surface of the wall.

10. The cartridge of claim 9, wherein each intermediate portion comprises a hygroscopic element, a dummy material, an air gap, an inert gas, or any combination thereof.

11. The cartridge of claim 1, further comprising a rotor configured to rotate the plurality of chambers about a rotation axis.

12. The cartridge of claim 1, further comprising:
a reagent enclosed by the wall of the at least one of the plurality of chambers; and
a lid coupled to the plurality of chambers and configured to seal the plurality of chambers.

13. The cartridge of claim 12, wherein the reagent comprises a lyophilized reagent.

14. The cartridge of claim 12, wherein the fluid is enclosed in one of the plurality of chambers that is external to the intermediate portion and external to the chamber enclosing the reagent.

15. A diagnostic assay system comprising:
a cartridge configured to receive a fluid and comprising a plurality of chambers and a rotor, wherein the rotor comprises a rotor port in fluid communication with at least one of the plurality of chambers;
a syringe system coupled to the cartridge and configured to provide the fluid to the rotor port, the syringe system comprising a syringe barrel having a barrel port and a syringe plunger configured to inject and withdraw the fluid;
a first actuator coupled to the cartridge and configured to rotate the rotor port into alignment with the barrel port;
a second actuator coupled to the syringe system and configured to translate the syringe plunger; and
a controller coupled to the first and second actuators and configured to detect one or more characteristics of the fluid based on an interaction of the fluid with one or more lyophilized reagents,
wherein one of the plurality of chambers comprises a wall enclosing a lyophilized reagent, and
wherein the wall comprises an intermediate portion disposed between an exterior surface and an interior surface of the wall.

16. A method of detecting one or more characteristics of a fluid in a diagnostic assay system, the method comprising:
forming a cartridge having a plurality of chambers and a rotor, wherein a first chamber of the plurality of chambers comprises a wall having an intermediate portion disposed between an exterior surface and an interior surface of the wall;
disposing a reagent within the first chamber; and
causing the fluid to interact with the reagent in the first chamber.

17. The method of claim 16, further comprising disposing a hygroscopic element, a dummy material, air, or an inert gas in the intermediate portion in order to extend an active period of interacting the fluid with the reagent.

18. The method of claim 16, further comprising detecting one or more characteristics of the fluid based on the interaction of the fluid with the reagent.

19. The method of claim 16, further comprising interacting the fluid with a second reagent in a second chamber of the plurality of chambers, wherein the second chamber comprises a wall having a hygroscopic element, a dummy material, an air gap, or an inert gas disposed between an exterior surface and an interior surface of the wall.

20. The method of claim 19, further comprising detecting one or more characteristics of the fluid based on the interaction of the fluid with the reagent and the second reagent.

21. The cartridge of claim 1, wherein the intermediate portion comprises an inert gas.

22. The cartridge of claim 21, wherein the inert gas comprises a noble gas, dinitrogen (N₂), carbon dioxide (CO₂), or a combination thereof.

23. The method of claim 16, wherein the intermediate portion comprises an inert gas.

24. The method of claim 23, wherein the inert gas comprises a noble gas, dinitrogen (N₂), carbon dioxide (CO₂), or a combination thereof.
